(19) United States
(12) Patent Application Publication
(10) Pub. No.: US 2004/0039019 A1
(43) Pub. Date: Feb. 26, 2004

(54) Selected anthranilaminde pyridinamides and their use as pharmaceutical agents

(75) Inventors: Andreas Huth, Berlin (DE); Martin Krueger, Berlin (DE); Ludwig Zorn, Berlin (DE); Stuart Ince, Berlin (DE); Karl-Heinz Thierauch, Berlin (DE); Andreas Menrad, Oranienburg (DE); Martin Haberey, Berlin (DE); Holger Hess-Stumpp, Berlin (DE)

Correspondence Address: MILLEN, WHITE, ZELANO & BRANIGAN, P.C., 2200 CLARENDON BLVD., SUITE 1400, ARLINGTON, VA, 22201, US

(73) Assignee: Schering AG, Berlin (DE)

(21) Appl. No.: 10/464,853

(22) Filed: Jun. 19, 2003

Related U.S. Application Data
(60) Provisional application No. 60/404,773, filed on Aug. 21, 2002.

(30) Foreign Application Priority Data
Jun. 19, 2002 (DE) 102 28 090.8

(51) Int. Cl. A61K 31/4709; A61K 31/4439; C 07D 4 1/02; C 07D 4 5/02

(52) U.S. Cl. 514/314; 514/337; 514/339; 546/159; 546/277.4; 546/283.1

(57) ABSTRACT

Selected anthranilamide pyridinamines of general formula I

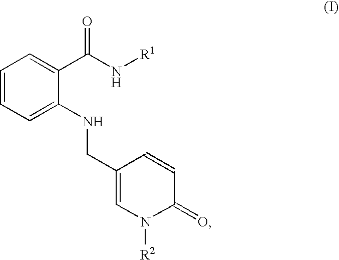

in which R¹ and R² have the meanings that are indicated in the description, as VEGFR-2 and VEGFR-3 inhibitors, their production and use as pharmaceutical agents for treating various diseases that are triggered by persistent angiogenesis, are described.

The invention relates to selected anthranilamide pyridinamines as VEGFR-2 and VEGFR-3 inhibitors, their production and use as pharmaceutical agents for treating diseases that are triggered by persistent angiogenesis.

Persistent angiogenesis can be the cause of various diseases, such as psoriasis; arthritis, such as rheumatoid arthritis, hemangioma, angiofibroma; eye diseases, such as diabetic retinopathy, neovascular glaucoma; renal diseases, such as glomerulonephritis, diabetic nephropathy, malignant nephrosclerosis, thrombic microangiopathic syndrome, transplant rejections and glomerulopathy; fibrotic diseases, such as cirrhosis of the liver, mesangial cell proliferative diseases and arteriosclerosis, or it can result in an aggravation of these diseases.

Persistent angiogenesis is induced by the factor VEGF via its receptor. So that VEGF can exert this action, it is necessary that VEGF bind to the receptor, and a tyrosine phosphorylation is induced.

Direct or indirect inhibition of the VEGF receptor (VEGF=vascular endothelial growth factor) can be used for treating such diseases and other VEGF-induced pathological angiogenesis and vascular permeable conditions, such as tumor vascularization. For example, it is known that the growth of tumors can be inhibited by soluble receptors and antibodies against VEGF.

Anthranilic acid amides that are used as pharmaceutical agents for treating psoriasis; arthritis, such as rheumatoid arthritis, hemangioma, angiofibroma; eye diseases, such as diabetic retinopathy, neovascular glaucoma; renal diseases, such as glomerulonephritis, diabetic nephropathy, malignant nephrosclerosis, thrombic microangiopathic syndrome, transplant rejections and glomerulopathy; fibrotic diseases, such as cirrhosis of the liver, mesangial cell proliferative diseases, arteriosclerosis, injuries to nerve tissue, and for inhibiting the reocclusion of vessels after balloon catheter treatment, in vascular prosthetics or after mechanical devices are used to keep vessels open, such as, e.g., stents, are known from WO 00/27819.

The known compounds are generally effective in the indications cited, but their effectiveness generally accompanies toxicity and an inferior compatibility of the medication.

There is therefore a desire, on the one hand, for more effective compounds, and, on the other hand, for more toxicologically harmless compounds, which, moreover, should also be more compatible.

It has now been found that compounds of general formula I

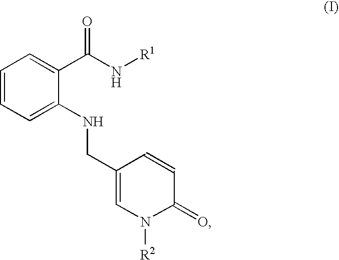

in which

R¹ stands for indazolyl, indolinyl, quinolinyl or for the group

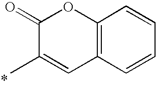

which optionally can be substituted in one or more places in the same way or differently with halogen, hydroxy, C₁-C₄-alkyl, C₁-C₄-alkoxy, halo-C₁-C₄-alkyl or with the group ═O or —OR³ or with cyano-C₁-C₃-alkyl,

R₂ stands for hydrogen or C₁-C₃-alkyl, and

R³ stands for hydrogen or C₁-C₄-alkyl, with the exception of the compounds in which R² stands for hydrogen or methyl, and R¹ simultaneously stands for unsubstituted indazolyl or quinolinyl, as well as the enantiomers, racemates, isomers and salts thereof, overcome the above-indicated drawbacks.

The compounds according to the invention prevent a tyrosine phosphorylation or stop persistent angiogenesis and thus the growth and propagation of tumors, whereby they are distinguished in particular by a slighter inhibition of isoforms of Cytochrome P 450 (2C9 and 2C19).

Pharmaceutical agents that are degraded by these isoforms are generally less toxic and better-tolerated.

Alkyl is defined in each case as a straight-chain or branched alkyl radical, such as, for example, methyl, ethyl, propyl, isopropyl, butyl, isobutyl, sec-butyl, pentyl, isopentyl, or hexyl, whereby C₁-C₄-alkyl radicals are preferred.

Alkoxy is defined in each case as a straight-chain or branched alkoxy radical, such as, for example, methyloxy, ethyloxy, propyloxy, isopropyloxy, butyloxy, isobutyloxy, or sec-butyloxy.

Halogen is defined in each case as fluorine, chlorine, bromine or iodine.

If an acid group is included, the physiologically compatible salts of organic and inorganic bases are suitable as salts, such as, for example, the readily soluble alkali salts and alkaline-earth salts as well as N-methyl-glucamine, dimethyl-glucamine, ethyl-glucamine, lysine, 1,6-hexadiamine, ethanolamine, glucosamine, sarcosine, serinol, tris-hydroxy-methyl-amino-methane, aminopropanediol, Sovak base, and 1-amino-2,3,4-butanetriol.

If a basic group is included, the physiologically compatible salts of organic and inorganic acids are suitable, such as hydrochloric acid, sulfuric acid, phosphoric acid, citric acid, tartaric acid, fumaric acid, i.a.

Those compounds of general formula I, in which

R¹ stands for indazolyl, indolinyl, quinolinyl or for the group

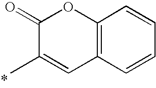

which optionally can be substituted in one or more places in the same way or differently with methyl, methoxy or with the group ═O or with cyanoethyl, and

R² stands for hydrogen, with the exception of the compounds in which R² stands for hydrogen, and R¹ simultaneously stands for unsubstituted indazolyl or quinolinyl, as well as the enantiomers, racemates, isomers and salts thereof, are especially effective.

The compounds of general formula I according to the invention also contain the possible tautomeric forms and comprise the E- or Z-isomers, or, if a chiral center is present, also the racemates and enantiomers.

The compounds according to the invention as well as their physiologically compatible salts prevent a tyrosine phosphorylation or stop persistent angiogenesis and thus the growth and propagation of tumors, whereby they are distinguished in particular by a slighter inhibition of isoforms of Cytochrome P 450 (2C9 and 2C19). Medication using the compounds according to the invention can therefore be done at no risk even without regard to pharmaceutical agents that are administered at the same time and that are degraded via these isoforms.

The compounds of formula I as well as their physiologically compatible salts can be used as pharmaceutical agents based on their inhibitory activity relative to the phosphorylation of the VEGF receptor. Based on their profile of action, the compounds according to the invention are suitable for treating diseases that are caused or promoted by persistent angiogenesis.

Since the compounds of formula I are identified as inhibitors of the tyrosine kinases KDR and FLT, they are suitable in particular for treating those diseases that are caused or promoted by persistent angiogenesis that is triggered via the VEGF receptor or by an increase in vascular permeability.

The subject of this invention is also the use of the compounds according to the invention as inhibitors of the tyrosine kinases KDR and FLT.

Subjects of this invention are thus also pharmaceutical agents for treating tumors or use thereof.

The compounds according to the invention can be used either alone or in a formulation as pharmaceutical agents for treating psoriasis, Kaposi's sarcoma; restenosis, such as, e.g., stent-induced restenosis, endometriosis, Crohn's disease, Hodgkin's disease, leukemia; arthritis, such as rheumatoid arthritis, hemangioma, angiofibroma; eye diseases, such as diabetic retinopathy, neovascular glaucoma; renal diseases, such as glomerulonephritis, diabetic nephropathy, malignant nephrosclerosis, thrombic microangiopathic syndrome, transplant rejections and glomerulopathy; fibrotic diseases, such as cirrhosis of the liver, mesangial cell proliferative diseases, arteriosclerosis, injuries to nerve tissue, and for inhibiting the reocclusion of vessels after balloon catheter treatment, in vascular prosthetics or after mechanical devices are used to keep vessels open, such as, e.g., stents, as immunosuppressive agents, for supporting scar-free healing, in senile keratosis and in contact dermatitis.

In treating injuries to nerve tissue, quick scar formation on the injury sites can be prevented with the compounds according to the invention, i.e., scar formation is prevented from occurring before the axons reconnect. A reconstruction of the nerve compounds was thus facilitated.

The formation of ascites in patients can also be suppressed with the compounds according to the invention. VEGF-induced edemas can also be suppressed.

Lymphangiogenesis plays an important role in lymphogenic metastasizing (Karpanen, T. et al., Cancere Res. Mar. 1, 2001, 61(5): 1786-90, Veikkola, T., et al., EMBO J. Mar. 15, 2001; 20(6): 1223-31).

The compounds according to the invention now also show excellent action as VEGFR kinase 3 inhibitors and are therefore also suitable as effective inhibitors of lymphangiogenesis.

By a treatment with the compounds according to the invention, not only a reduction in the size of metastases but also a reduction in the number of metastases is achieved.

Such pharmaceutical agents, their formulations and uses, are also subjects of this invention.

The invention thus also relates to the use of the compounds of general formula I for the production of a pharmaceutical agent for use as or for treatment of psoriasis, Kaposi's sarcoma; restenosis, such as, e.g., stent-induced restenosis, endometriosis, Crohn's disease, Hodgkin's disease, leukemia; arthritis, such as rheumatoid arthritis, hemangioma, angiofibroma; eye diseases, such as diabetic retinopathy, neovascular glaucoma; renal diseases, such as glomerulonephritis, diabetic nephropathy, malignant nephrosclerosis, thrombic microangiopathic syndrome, transplant rejections and glomerulopathy; fibrotic diseases, such as cirrhosis of the liver, mesangial cell proliferative diseases, arteriosclerosis, injuries to nerve tissue, and for inhibiting the reocclusion of vessels after balloon catheter treatment, in vascular prosthetics or after mechanical devices are used to keep vessels open, such as, e.g., stents, as immunosuppressive agents, for supporting scar-free healing, in senile keratosis and in contact dermatitis.

The formation of ascites in patients can also be suppressed with the compounds according to the invention. VEGF-induced edemas can also be suppressed.

To use the compounds of formula I as pharmaceutical agents, the latter are brought into the form of a pharmaceutical preparation, which in addition to the active ingredient for enteral or parenteral administration contains suitable pharmaceutical, organic or inorganic inert carrier materials, such as, for example, water, gelatin, gum arabic, lactose, starch, magnesium stearate, talc, vegetable oils, polyalkylene glycols, etc. The pharmaceutical preparations can be present in solid form, for example as tablets, coated tablets, suppositories, capsules, or in liquid form, for example as solutions, suspensions or emulsions. They also contain, moreover, adjuvants such as preservatives, stabilizers, wetting agents or emulsifiers, salts for changing osmotic pressure or buffers.

For parenteral administration, especially injection solutions or suspensions, especially aqueous solutions of the active compounds in polyhydroxyethoxylated castor oil, are suitable.

As carrier systems, surface-active adjuvants such as salts of bile acids or animal or plant phospholipids, but also mixtures thereof as well as liposomes or components thereof can also be used.

For oral administration, especially tablets, coated tablets or capsules with talc and/or hydrocarbon vehicles or binders, such as for example, lactose, corn starch or potato starch, are suitable. The administration can also be carried out in liquid form, such as, for example, as juice, to which optionally a sweetener or, if necessary, one or more flavoring substances, is added.

The dosage of the active ingredients can vary depending on the method of administration, age and weight of the patient, type and severity of the disease to be treated and similar factors. The daily dose is 0.5-1000 mg, preferably 50-200 mg, whereby the dose can be given as a single dose to be administered once or divided into 2 or more daily doses.

The above-described formulations and forms for dispensing are also subjects of this invention.

Production of the Compounds According to the Invention

The following examples explain the production of the compounds according to the invention without the scope of the claimed compounds being limited to these examples.

The production of the compounds according to the invention is carried out according to methods that are known in the art, by an amine of general formula II

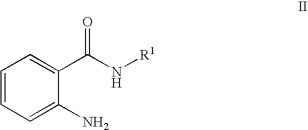

in which R¹ has the meaning indicated in general formula I, being alkylated, and then the amine of general formula II being subjected to reductive alkylation with aldehydes or ketones, whereby it is reacted in the presence of a reducing agent, such as, for example, sodium cyanoborohydride, in a suitable inert solvent, such as, for example, ethanol, at temperatures from 0° C. up to the boiling point of the solvent. An addition of acids such as glacial acetic acid may prove advantageous.

If a start is made from a primary amino group, a reaction can be performed optionally in succession with two different carbonyl compounds, whereby mixed derivatives are obtained [literature, e.g., Verardo et al. Synthesis (1993), 121; Synthesis (1991), 447; Kawaguchi, Synthesis (1985), 701; Micovic et al. Synthesis (1991), 1043].

There are also methods to react 1,2-dichloroethane with triacetoxy borohydride (J. Org. Chem. 1996, 3849). It may be advantageous first to form the Schiff base by reaction of the aldehyde with the amine in solvents such as ethanol or methanol, optionally with the addition of adjuvants such as glacial acetic acid, and then to add only a reducing agent, such as, e.g., sodium cyanoborohydride. The Schiff base can also be isolated, however, and then reduced to form amine in solvents such as diethyl ether or tetrahydrofuran with reducing agents, such as, for example, lithium aluminum hydride.

For the production of amides, a start is made from compounds of general formula

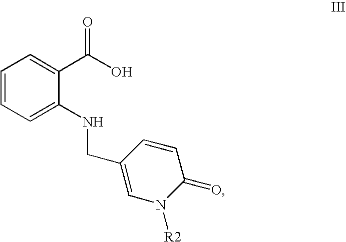

whereby for this purpose, the processes that are known from peptide chemistry are available. For example, the corresponding acid in aprotic polar solvents, such as, for example, dimethylformamide, can be reacted via an activated acid derivative that can be obtained, for example, with hydroxybenzotriazole and a carbodiimide, such as, for example, diisopropylcarbodiimide, at temperatures of between 0° C. and the boiling point of the solvent, preferably at 80° C. with the amine. Also, the method of using O-(7-azabenzotriazol-1-yl)-1,1,3,3-tetramethyluronium hexafluorophosphate (HATU) for the activation provides good yields. As a base, for example, N-methylmorpholine is used, and as a solvent, for example, dimethylformamide is used. The reaction preferably proceeds at room temperature. For the amide formation, the process with the mixed acid anhydride, imidazolide or azide can also be used. A prior protection of an additional amino group, for example as an amide, is not necessary in all cases, but can advantageously influence the reaction.

Subjects of this application are thus also compounds of general formulas II and III

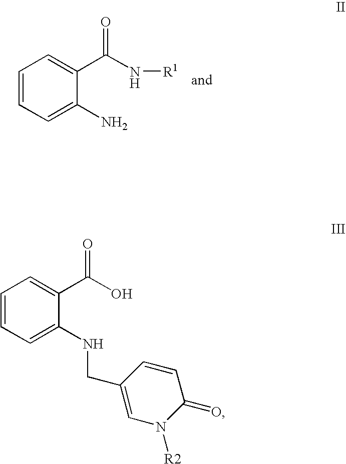

in which R¹ and R² have the meanings that are indicated in general Formula I, as intermediate products for the production of the compounds of general formula I.

### EXAMPLE 1.0

Production of N-(2-Oxo-2,3-dihydro-1H-indol-6-yl)-2-[(6-oxo-1,6-dihydro-pyridin-3-ylmethyl)-amino]-benzamide

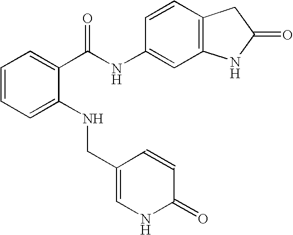

4.5 g (16.9 mmol) of 2-amino-N-(2-oxo-2,3-dihydro-1H-indol-6-yl)-benzamide is introduced into 90 ml of absolute methanol together with 2.5 g (20.3 mmol) of pyrid-2-one-5-carboxaldehyde, and it is mixed with 2.5 ml of glacial acetic acid. The feedstock is stirred overnight at room temperature. It is cooled to 4° C., mixed twice with 750 mg of sodium cyanoborohydride each, and stirred for 24 hours. The solid is suctioned off and washed with methanol and water. It is then absorptively precipitated with saturated sodium bicarbonate solution and suctioned off again. The solid is dried. 5.16 g (82% of theory) of N-(2-oxo-2,3 -dihydro-1H-indol-6-yl)-2-[(6-oxo-1,6-dihydro-pyridin-3-ylmethyl)-amino]-benzamide is obtained.

Similarly produced are also:

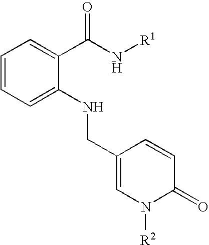

| | | | Melting Point |
|---|---|---|---|
| Example No. | R¹ | R² | [° C.] |
| 1.1 | 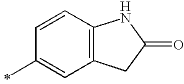 | H | |
| 1.2 | 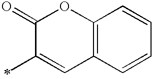 | H | |
| 1.3 | 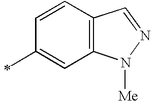 | H | |
| 1.4 | 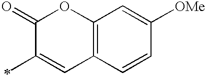 | H | |
| 1.5 | 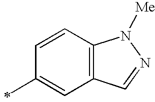 | H | |
| 1.6 | 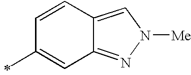 | H | |
| 1.7 | 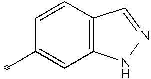 | H | |
| 1.8 | 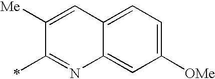 | H | |

### EXAMPLE 2.0

Production of N-(1-Cyanomethyl-1H-indazol-6-yl)-2-[(6-oxo-1,6-dihydro-pyridin-3-ylmethyl)-amino]-benzamide

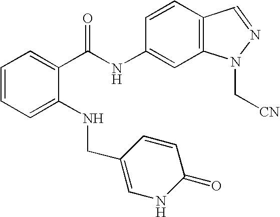

317 mg (1.3 mmol) of N-(1H-indazol-6-yl)-2-[(6-oxo-1,6-dihydro-pyridin-3-ylmethyl)-amino]-benzamide is mixed in 15 ml of dimethylformamide with 280 mg (1.6 mmol) of (6-amino-indazol-1-yl)-acetonitrile, 330 mg of N-methylmorpholine and 591 mg (1.55 mmol) of O-(7-azabenzotriazol-1-yl)-1,1,3,3-tetramethyluroniumhexafluoro-phosphate (HATU), and it is stirred for 3 hours at room temperature. The feedstock is then evaporated to the dry state in a vacuum and distributed in 30 ml of ethyl acetate and 30 ml of water, whereby it is also suctioned off from the solid. The ethyl acetate phase is dried, filtered and concentrated by evaporation. The residue is purified on a Flashmaster column with a gradient of methylene chloride:ethanol=100:0 to 90:10 as an eluant. 286 mg/55% of theory) of N-(1-cyanomethyl-1H-indazol-6-yl)-2-[(6-oxo-1,6-dihydro-pyridin-3-ylmethyl)-amino]-benzamide is obtained.

### EXAMPLE 2.1

Production of N-(2-Cyanomethyl-2H-indazol-6-yl)-2-[(6-oxo-1,6-dihydro-pyridin-3-ylmethyl)-amino]-benzamide

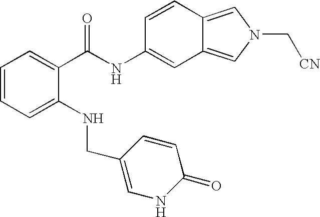

N-(2-Cyanomethyl-2H-indazol-6-yl)-2-[(6-oxo-1,6-dihydro-pyridin-3-ylmethyl)-amino]-benzamide is produced analogously to Example 2.0.

Production of Starting Materials

Process Variant A

If the production of the intermediate compounds is not described, the latter are known or can be produced analogously to known compounds or to processes that are described here.

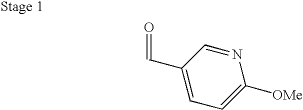

2-Methoxypyridine-5-carbaldehyde is a commercially available product (Aldrich)

ii)

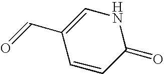

2-Pyridone-5carbaldehyde is produced, e.g., according to Hoppe Seylers Zeitschrift für physiologische Chemie [Hoppe Seyler's Journal for Physiological Chemistry], Vol. 325, p. 239, (1961).

Process Variant B

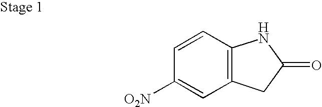

5-Nitro-1,3-dihydro-indol-2-one is produced according to R. T. Courts, J. Org. Chem. 48, 3747, 1970).

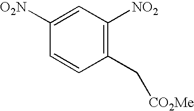

22.6 g (100 mmol) of 2,4-dinitrophenylacetic acid is dissolved in a mixture of 200 ml of methanol and 830 ml of toluene, mixed at room temperature with 83 ml of trimethylsilyldiazomethane (2 molar in toluene; 166 mmol) and stirred for 3 hours at room temperature. After concentration by evaporation to the dry state and drying at 70° C. in a vacuum, 24 g (100 % of theory) of 2,4-dinitrophenyl-acetic acid methyl ester is obtained.

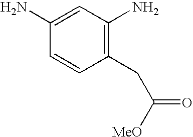

26.6 g (83 mmol) of 2,4-dinitrophenylacetic acid methyl ester is hydrogenated in 500 ml of ethanol with 5.4 g of palladium/carbon (10%) under 1 bar of hydrogen for 1.5 hours at room temperature. After catalyst is filtered out, it is concentrated by evaporation. 18.8 g (94% of theory) of 2,4-diaminophenylacetic acid methyl ester is obtained

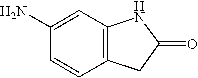

18.8 g (104 mmol) of 2,4-diaminophenylacetic acid methyl ester in 500 ml of absolute toluene is mixed at 4° C. with 52 ml of 2-molar solution of trimethyl aluminum in toluene, and then it is heated for 60 minutes to 60° C. After cooling, it is set at pH 7 with 2N sodium hydroxide solution, filtered on diatomaceous earth and extracted from ethyl acetate. The organic phase is washed, dried, filtered and concentrated by evaporation. 8.7 g (55% of theory) of 6-amino-1,3-dihydro-indol-2-one is obtained.

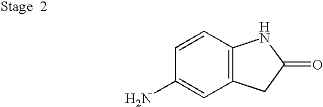

356 mg of 5-nitro-1,3-dihydro-indol-2-one is hydrogenated in 30 ml of tetrahydrofuran:ethanol=1:1 with 400 mg palladium on carbon (10%) at room temperature and normal pressure for 1 hour. After catalyst is suctioned off on diatomaceous earth and after concentration by evaporation, 320 mg (100% of theory) of 5-amino-1,3-dihydro-indol-2-one is obtained.

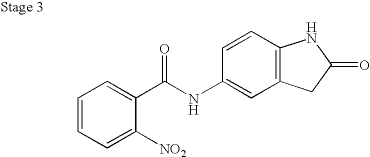

In 1 ml of dimethylacetamide, 320 mg of 5-amino-1,3-dihydro-indol-2-one is dissolved and mixed drop by drop with 371 mg (2 mmol) of 2-nitrobenzoyl chloride, whereby a slight heating occurs. After stirring overnight at room temperature, it is concentrated by evaporation in a vacuum, and the residue is taken up in ethyl acetate and water. The suctioning off of an insoluble solid provides 130 mg (21.9% of theory) of 2-nitro-N-(2-oxo-2,3-dihydro-1H-indol-5-yl)-benzamide. After shaking out, the organic phase is washed, filtered and concentrated by evaporation, and 400 mg (67% of theory) of 2-nitro-N-(2-oxo-2,3-dihydro-1H-indol-5-yl)-benzamide with a melting point of 265° C. is obtained once more.

Similarly produced is also 2-nitro-N-(2-oxo-2,3-dihydro-1H-indol-6-yl)-benzamide with a melting point >300° C.

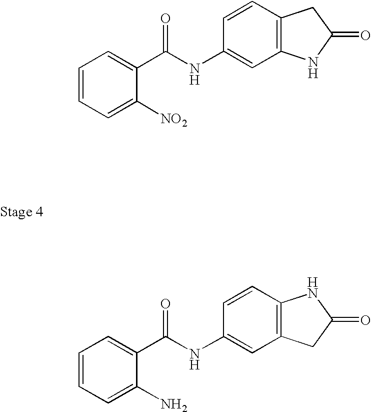

Similarly produced to Stage 2 is 2-amino-N-(indol-2-on-5-yl)benzoic acid amide with a melting point of 219° C.

Similarly produced to Stage 2 of this example is 2-amino-N-(indol-2-on-6-yl)benzoic acid amide with a melting point of 230° C.

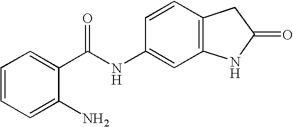

Process Variant C

Production of 2-Amino-N-(7-methoxy-2-oxo-2H-chromen-3-yl)-benzamide

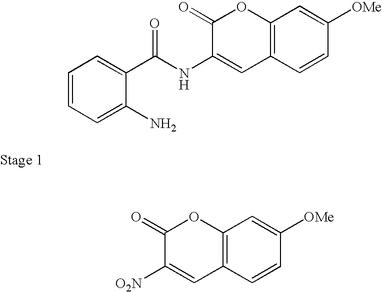

13 g (85.4 mmol) of 2-hydroxy-4-methoxybenzaldehyde is heated in 300 ml of toluene with 9.8 g (102.5 mmol) of n-propylamine hydrochloride and 11.5 ml (102.5 mmol) of nitroacetic acid ethyl ester for 15 hours in a water separator. 3 ml of nitroacetic acid ethyl ester is then added again and boiled for another 5 hours in a water separator. After cooling, it is diluted with ethyl acetate and shaken out with water. The ethyl acetate phase is dried, filtered and concentrated by evaporation. The residue is chromatographed on silica gel with methylene chloride as an eluant. 6.14 g (33% of theory) of 3-nitro-7-methoxy-chromen-2-one is obtained.

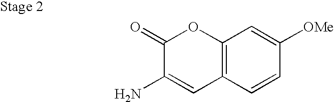

In a way similar to Stage 2 from Example B, 3-amino-7-methoxy-chromen-2-one is produced from 3-nitro-7-methoxy-chromen-2-one in ethanol.

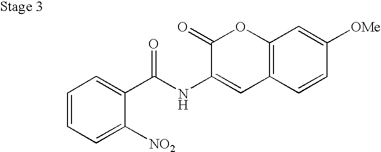

In a way similar to Stage 3 from Example B, 2-nitro-N-(7-methoxybenzopyran-2-on-3-yl)benzoic acid amide is produced from 2-nitrobenzoyl chloride and 3-amino-7-methoxy-chromen-2-one [and] 2-nitro-N-(7-methoxy-2-oxo-2H-chromen-3-yl)-benzamide. 2-Amino-N-(7-methoxy-2-oxo-2H-chromen-3-yl)-benzamide

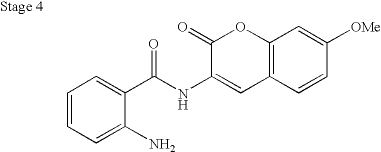

In a way similar to Stage 2 of Example B, 2-amino-N-(7-methoxy-2-oxo-2H-chromen-3-yl)-benzamide is produced from 2-nitro-N-(7-methoxy-2-oxo-2H-chromen-3-yl)-benzamide in ethanol:tetrahydrofuran=5:2.

Similarly produced are also the following intermediate compounds:

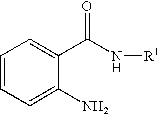

| | | Melting Point |
|---|---|---|
| Example No. | R¹ | [° C.] |
| i | 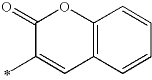 | |
| ii | 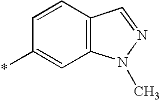 | |
| iii | 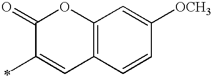 | |
| iv | 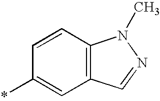 | |
| v | 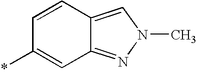 | |
| vi | 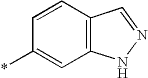 | |
| vii | 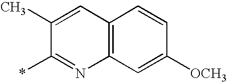 | |

Process Variant D

Stage 1

Analogously to Example 1,2-[(6-oxo-1,6-dihydro-pyridin-3-ylmethyl)-amino]-benzoic acid methyl ester is produced from anthranilic acid ester and 2-pyridone-5-carbaldehyde in a 72% yield.

Stage 2

450 mg (1.74 mmol) of 2-[(6-oxo-1,6-dihydro-pyridin-3-ylmethyl)-amino]-benzoic acid methyl ester is mixed in 20 ml of ethanol with 4 ml of 1N sodium hydroxide solution, and it is heated for 1 hour to a bath temperature of 120° C. After the ethanol is distilled off in a vacuum, it is diluted with 10 ml of water and set at pH 7 with 2N hydrochloric acid. The precipitated product is suctioned off. 242 mg (57% of theory) of a yield of 2-[(6-oxo-1,6-dihydro-pyridin-3-ylmethyl)-amino]-benzoic acid is obtained.

The sample applications below explain the biological action and the use of the compounds according to the invention without the latter being limited to the examples.

Solutions Required for the Tests

Stock solutions

Stock solution A: 3 mmol of ATP in water, pH 7.0 (−70° C.)

Stock solution B: g-33P-ATP 1 mCi/100 μl

Stock solution C: poly-(Glu4Tyr) 10 mg/ml in water

Solution for dilutions

Substrate solvent: 10 mmol of DTT, 10 mmol of manganese chloride, 100 mmol of magnesium chloride

Enzyme solution: 120 mmol of tris/HCl, pH 7.5, 10 μM of sodium vanadium oxide

Sample Application 1

Inhibition of the KDR- and FLT-1 Kinase Activity in the Presence of the Compounds According to the Invention

In a microtiter plate (without protein binding) that tapers to a point, 10 μl of substrate mix (10 μl of volume of ATP stock solution A +25 μCi of g-33P-ATP (about 2.5 μl of stock solution B)+30 μl of poly-(Glu4Tyr) stock solution C+1.21 ml of substrate solvent), 10 μl of inhibitor solution (substances corresponding to the dilutions, 3% DMSO in substrate solvent as a control) and 10 μl of enzyme solution (11.25 μg of enzyme stock solution (KDR or FLT-1 kinase) are added at 4° C. in 1.25 ml of enzyme solution (dilute). It is thoroughly mixed and incubated for 10 minutes at room temperature. Then, 10 μl of stop solution (250 mmol of EDTA, pH 7.0) is added, mixed, and 10 μl of the solution is transferred to a P 81 phosphocellulose filter. Then, it is washed several times in 0.1 M phosphoric acid. The filter paper is dried, coated with Meltilex and measured in a microbeta counter.

The IC50 values are determined from the-inhibitor concentration, which is necessary to inhibit the phosphate incorporation to 50% of the uninhibited incorporation after removal of the blank reading (EDTA-stopped reaction).

The results of the kinase inhibition IC50 in μM are presented in the table below:

| | VEGFR II (KDR) |
|---|---|
| Example No. | [μM] |
| 1.0 | 0.05 |

Sample Application 2

Cytochrome P450 Inhibition

The Cytochrome P450 inhibition was performed according to the publication of Crespi et al. (Anal. Biochem., 248, 188-190 (1997)) with use of baculovirus/insect cell-expressed, human Cytochrome P 450 isoenzymes (1A2, 2C9, 2C19, 2D6, 3A4).

The results are presented in the following table.

Inhibition of the Cytochrome P450 Isoenzymes (IC50, μM)

| Cytochrome | | | | | |
|---|---|---|---|---|---|
| P450 | | | | | |
| Isoenzyme | 1A2 | 2C9 | 2C19 | 2D6 | 3A4 |
| Example | 5.2 | 0.2 | 0.05 | >30 | 3.6 |
| 2.54 of WO | | | | | |
| 00/27819 | | | | | |
| Example 1.0 | >30 | 23 | >30 | >30 | >30 |

The superior action of the compounds according to the invention compared to the known compounds can be seen clearly from the result.

Without further elaboration, it is believed that one skilled in the art can, using the preceding description, utilize the present invention to its fullest extent. The preceding preferred specific embodiments are, therefore, to be construed as merely illustrative, and not limitative of the remainder of the disclosure in any way whatsoever.

In the foregoing and in the examples, all temperatures are set forth uncorrected in degrees Celsius and, all parts and percentages are by weight, unless otherwise indicated.

The entire disclosures of all applications, patents and publications, cited herein and of corresponding German application No. 102 28 090.8, filed Jun. 19, 2002, and U.S. Provisional Application Serial No. 60/404,773, filed Aug. 21, 2002, are incorporated by reference herein.

The preceding examples can be repeated with similar success by substituting the generically or specifically described reactants

1. Compounds of general formula I 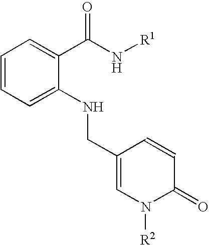
in which
R¹ stands for indazolyl, indolinyl, quinolinyl or for the group 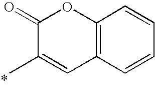
which optionally can be substituted in one or more places in the same way or differently with halogen, hydroxy, C₁-C₄-alkyl, C₁-C₄-alkoxy, halo-C₁-C₄-alkyl or with the group ═O or —OR³ or with cyano-C₁-C₃-alkyl,
R² stands for hydrogen or C₁-C₃-alkyl, and
R³ stands for hydrogen or C₁-C₄-alkyl, with the exception of the compounds in which R² stands for hydrogen or methyl, and R¹ simultaneously stands for unsubstituted indazolyl or quinolinyl, as well as the enantiomers, racemates, isomers and salts thereof:

2. Compounds of general formula I, in which
R¹ stands for indazolyl, indolinyl, quinolinyl or for the group 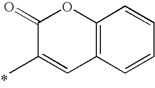
which optionally can be substituted in one or more places in the same way or differently with methyl, methoxy or with the group ═O or with cyanoethyl, and
R² stands for hydrogen, with the exception of the compounds in which R stands for hydrogen, and R¹ simultaneously stands for unsubstituted indazolyl or quinolinyl, as well as the enantiomers, racemates, isomers and salts thereof.

3. Pharmaceutical agents that contain at least one compound according to claims 1 and 2.

4. Pharmaceutical agents according to claim 3 for treating tumors, psoriasis, Kaposi's sarcoma, restenosis, stent-induced restenosis, endometriosis, Crohn's disease, Hodgkin's disease, leukemia, arthritis, rheumatoid arthritis, hemangioma, angiofibroma, eye diseases, diabetic retinopathy, neovascular glaucoma, renal diseases, glomerulonephritis, diabetic nephropathy, malignant nephrosclerosis, thrombic microangiopathic syndrome, transplant rejections, glomerulopathy, fibrotic diseases, cirrhosis of the liver, mesangial cell proliferative diseases, arteriosclerosis, injuries to nerve tissue, and for inhibiting the reocclusion of vessels after balloon catheter treatment, in vascular prosthetics or after the use of stents to keep vessels open, and of immune diseases as immunosuppressive agents, and for supporting scar-free healing in senile keratosis and in contact dermatitis.

5. Compounds according to claims 1 and 2 and pharmaceutical agents, according to claims 3 and 4, with suitable formulations and vehicles.

6. Use of the compounds of formula I, according to claims 1 and 2, as inhibitors of the tyrosine kinases KDR and FLT.

7. Use of the compounds of general formula I, according to claims 1 and 2, in the form of a pharmaceutical preparation for enteral, parenteral and oral administration.

8. Use of the compounds of general formula I, according to claims 1 and 2, as VEGFR-kinase 3 inhibitors in lymphangiogenesis.

9. Use of the compounds of general formula I, according to claims 1 and 2, for suppressing the formation of ascites and for suppressing VEGF-induced edemas.

10. Compounds of general formulas II and III 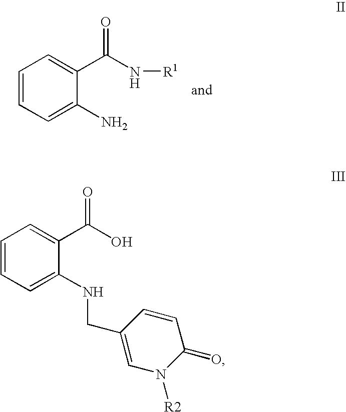
in which R¹ and R², which have the meanings indicated in general formula I, as intermediate products for the production of compounds of general formula I.
